(19) United States
(12) Patent Application Publication
(10) Pub. No.: US 2021/0233629 A1
(43) Pub. Date: Jul. 29, 2021

(54) ELECTRONIC MEDICAL RECORD SYSTEM PROVIDING CROSS-PATIENT DATA POPULATION AND DISPLAY

(71) Applicant: LANKENAU INSTITUTE FOR MEDICAL RESEARCH, WYNNEWOOD, PA (US)

(72) Inventor: Michelle Gray, King of Prussia, PA (US)

(21) Appl. No.: 17/157,554

(22) Filed: Jan. 25, 2021

Related U.S. Application Data
(60) Provisional application No. 62/966,320, filed on Jan. 27, 2020.

(51) Int. Cl. G16H 10/60; G16H 50/70; G16H 15/00; G06F 16/27; G06F 40/186

(52) U.S. Cl. CPC G16H 10/60; G16H 50/70; G16H 40/20; G06F 16/275; G06F 40/186; G16H 15/00

(57) ABSTRACT

An electronic medical records system providing a graphical user interface for receipt of data into a shared “chart” configured to receive data associated with multiple associated patients (e.g., mother and baby). The system is structured to provide a link from the shared chart to the electronic medical records of individual patients associated with the shared chart, and to provide data sharing and/or automated population of data from the shared chart into multiple linked electronic medical records of multiple patients in an appropriate selective fashion. The shared chart may be structured to gather information according to particular clinical workflows for multiple patients, and on a single interface window displayable on a single display screen. The system avoids duplicative data entry and associated errors while ensuring that the both related charts are up to date, and allows for compact data input and display relative to available physical space on a display device.

### CROSS-REFERENCE TO RELATED APPLICATION

This application claims the benefit of priority, under 35 U.S.C. § 119(e), of U.S. provisional patent application No. 62/966,320, filed Jan. 27, 2020, the entire disclosure of which is hereby incorporated herein by reference.

### FIELD OF THE INVENTION

The present invention relates generally to electronic medical record systems used for storing clinically-derived medical information about patients in a healthcare setting, and more particularly to an improved graphical user interface and method for gathering and managing information within such electronic medical record systems.

### DISCUSSION OF RELATED ART

Electronic medical record (EMR) systems offer several advantages, including reduced costs of creating, storing, and accessing the records. EMR systems typically use database storing records for individual patients. The values populating the data elements of the records and fields are typically collected by healthcare providers and entered into the EMR system to provide a complete record of the patient's medical treatment. Such records are subject to the Health Insurance Portability and Accountability Act (HIPAA), and are generally maintained in a secured fashion, such that access to those records is generally limited. Thus, electronic medical record systems facilitate the collection of all relevant information for a particular patient in a single system to provide robust medical records on an individual, patient-by-patient basis.

However, there are limited circumstances in which care of multiple patients is interrelated, and information may be relevant or shared across multiple individual patients. An example of such a circumstance is mother/child couplet care, following birth. According to the typical model, individual and separate electronic medical records (and/or “charts”) are created for both the mother and the baby. Certain activities, such as breastfeeding and related clinical observations, involve both the mother and the child. Accordingly, such systems provide certain information and/or observations to be first entered into the mother's chart/EMR record, then subsequently for the same information/observations to be entered into the child's chart/EMR record. Even in EMR systems that provide links permitting a healthcare provider to more easily navigate/switch between mother/child or other related charts, the relevant clinical information/observation data is not shared across the charts/records of the multiple patients by the system. Accordingly, duplicative manual data entry is required. This involves an operator/healthcare provider's opening/entering into multiple related charts and to repeatedly enter the relevant data, which is tedious and time consuming.

Further, such repeated recordation of relevant clinical data introduces additional opportunities for human error in data entry creating mismatches of data elements across both chart, errors in failing to record complete data for all related patients, and the like. Further still, data entry is disjoined and segmented in that a single mother/child process is segmented on the one hand from the perspective of the mother in the mother's EMR record, then on the other hand from the perspective of the child in the child's EMR record. For example, the segmentation of breastfeeding documentation across multiple patient EMRs and associated errors can prevent a hospital from achieving “Baby Friendly” status, which is advantageous to the hospital and ultimately to the patients, because attainment of such status requires months of documentation proof, which can be thwarted by incomplete and erroneous record keeping.

What is needed is an improved electronic medical records system that provides for data sharing across electronic medical records of related patients to and/or provides for entry of clinical observations in streamlined per-process fashion (across multiple patients), rather than in the conventional, segmented per-patient fashion, so as to reduce the burden on healthcare providers by avoiding duplicative entry of data for multiple patients, and to reduce or eliminate associated data entry errors and omissions.

### SUMMARY

The present invention provides a system and method providing an improved electronic medical records (EMR) system. The improved EMR system provides a graphical user interface for receipt of data from a healthcare provider into a shared “chart” configured to receive data associated with multiple related/interconnected patients, and a link from the shared chart to the electronic medical records of individual patients. Further, the EMR system provides for data sharing and/or automated population of data from the shared chart into multiple linked electronic medical records of multiple patients in an appropriate selective fashion. Preferably, the shared chart is arranged to gather information in a manner that is workflow specific, corresponding to a particular clinical workflow, involving data collection/input for multiple related/interconnected patients (such as a mother and one or more babies) in a single graphical user interface window displayable in its entirety on a single display screen.

More particularly, the EMR system provides a graphical user interface (GUI) acting as a shared “chart”, whereby a healthcare provider or other user can input data into the system via the shared chart to record clinical observations in a streamlined fashion, e.g., for breastfeeding activity, across multiple patients, e.g., a mother and child. Preferably, the GUI is constructed to display prompts for and/or receive data entry in a per-process fashion that logically corresponds to a clinical process/workflow (involving multiple patients), rather than on a per-patient basis that involves only a single patient.

Further, the system provides for a linking of the shared chart to related charts/records of involved patients (e.g., mother and child), and for data sharing among the shared and related electronic medical records, such that data entered manually into the shared chart by a healthcare provider, etc. is automatedly populated by the system into the respective linked related charts/electronic medical records of each individual patient (e.g., mother and child) in automated fashion by the EMR system. The data is populated into the respective linked/related charts selectively, such that each related chart receives only the data relevant to each chart, but also so that certain information is populated into more than 1 related chart in automated fashion.

For example, the GUI for the shared chart may be constructed to document a breastfeeding process (involving a mother and child) such that patient activity is documented on the mother's chart, and intake activity is document on the baby's chart, along with the multitude of other lactation criteria such as pain on the mother's part, LATCH score, infant satiety and feeding issues on the baby's chart.

In this manner, the improved EMR system reduces the data-entry burden on healthcare providers by avoiding duplicative entry of data for multiple patients, and reduce or eliminates associate data entry errors and omissions, while also ensure that the related (e.g., mother and child) charts are “in sync” and equally up to date, so that data provided in one chart/EMR record is not inadvertently omitted from the other chart/EMR record.

### BRIEF DESCRIPTION OF THE FIGURES

An understanding of the following description will be facilitated by reference to the attached drawings, in which:

FIG. 1 is a system diagram showing an exemplary network computing environment in which the present invention may be employed;

FIG. 2 is a simplified block diagram of an improved electronic medical record system in accordance with an exemplary embodiment of the present invention;

FIGS. 3A-3B are flow diagrams illustrating an exemplary method for promoting rendering of medical care in compliance with predetermined care protocols and for accurate logging of same;

FIGS. 4A-4F are plan views of exemplary graphical user interface windows of a conventional electronic medical records system of the prior art; and

FIG. 5 is a plan view of a graphical user interface window of an exemplary graphical user interface window of an improved electronic medical record system in accordance with the present invention.

### DETAILED DESCRIPTION

The present invention provides an improved EMR system configured to display a workflow-specific graphical user interface for receipt of data from a healthcare provider into a shared “chart”, and provide a linking from the shared chart to the electronic medical records of individual patients. Further, the EMR system provides for data sharing and/or automated population of data from the shared chart into multiple linked electronic medical records of multiple patients in an appropriate selective fashion. An exemplary embodiment of the present invention is discussed below for illustrative purposes.

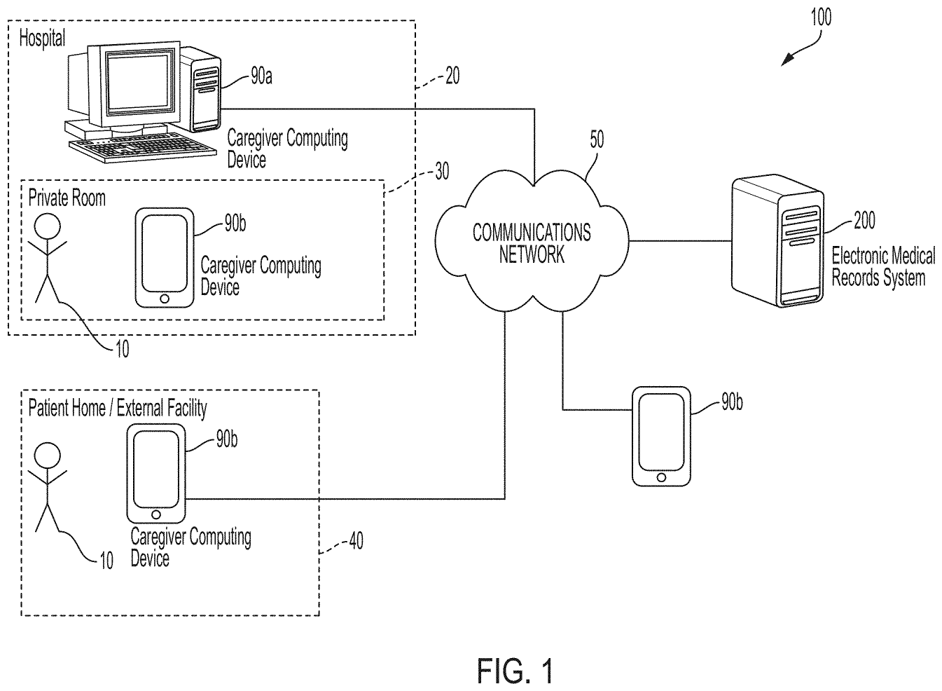

The present invention may be understood with reference to the exemplary simplified network environment 100 of FIG. 1. As shown in FIG. 1, the exemplary network environment 100 includes caregiver computing devices used by nurses/healthcare providers to enter data into patient medical records of an electronic medical records system, such as desktop computing device 90a and mobile computing devices 90b. Any suitable computing devices may be used for the purposes described herein. By way of example, the desktop computing device 90a may be a personal computer (PC) or the like that includes conventional hardware and software and is able to communicate with an electronic medical records (EMR) system 200 for the purposes described herein. Similarly, each mobile computing device 90b may be a smartphone, a tablet computer, or the like that includes conventional hardware and software and is able to communicate with the electronic medical records (EMR) system 200 for the purposes described herein. By way of example, these desktop computing device 90a may be disposed within a hospital 20, e.g., at a nurse's station, and the mobile computing device 90b may be transportable and may be used within a private room 30 of a patient 10 or elsewhere within the hospital 20, or at a patient's home or other care facility 40 outside the hospital 20, or elsewhere.

The exemplary network environment 100 also includes an improved EMR system 200 in accordance with the present invention. In this exemplary embodiment, the EMR system 200 is operatively connected to the computing devices 90a, 90b via a communications network 50, such as the Internet and/or a Virtual Private Network (VPN) connection. Hardware and software for enabling communication of data by such devices via such communications networks are well known in the art and beyond the scope of the present invention, and thus are not discussed in detail herein.

The exemplary network environment 100 includes an improved EMR system 200. The EMR system 200 may be similar to conventional EMR systems, such as the Cerner EMR system commercially available from Cerner Corporation of North Kansas City, Mississippi, in many respects. As known in the art, conventional EMR systems allow a user to enter data (such as patient identity information, health history information, blood test results and other records, clinical observations, etc.) into the system and to have such data added to and stored as part of an electronic medical record specific to a particular patient. As such, an EMR record for a patient is a collection of information describing the medical history of that patient. A patient's record may be managed by a variety of sources including a clinical facility, such as a hospital. The EMR record is essentially a collection of data stored in a data store, such as a database, and the database entries may be created by filling out an electronic form presented via a graphical user interface/window displayed by the EMR system. Various EMR systems and their operation are known in the art, and thus are not described in further detail herein.

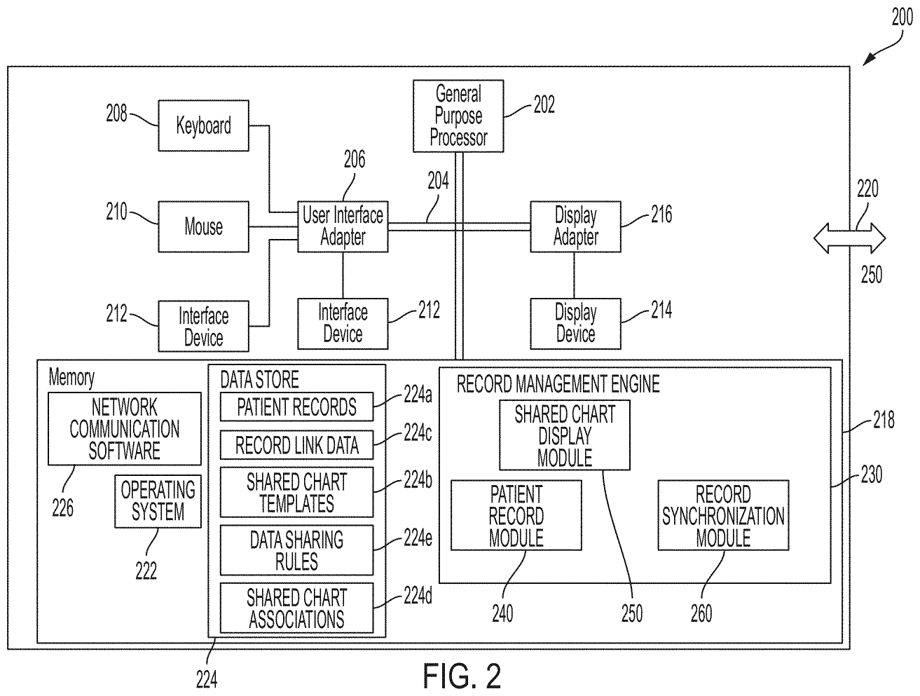

The improved EMR system 200 may include some or all of the structure and functionality typical of conventional EMR systems, but further includes additional structure and functionality in accordance with the present invention, as described herein. FIG. 2 is a block diagram showing an exemplary improved Electronic Medical Records (EMR) system 200 in accordance with an exemplary embodiment of the present invention. This EMR System 200 is a special-purpose computer system that includes conventional computing hardware storing and executing both conventional software enabling operation of a general purpose computing system, such as operating system software 222, network communications software 226, and specially-configured computer software for configuring the general purpose hardware as a special-purpose computer system for carrying out at least one method in accordance with the present invention. By way of example, the communications software 226 may include conventional web server software, and the operating system software 222 may include iOS, Android, Windows, Linux software.

Accordingly, the exemplary EMR system 200 of FIG. 2 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The EMR system 200 may communicate with other computers or networks of computers, for example via a communications channel, network card or modem 220. The EMR system 200 may be associated with such other computers in a local area network (LAN) or a wide area network (WAN), and may operate as a server in a client/server arrangement with another computer, etc. Such configurations, as well as the appropriate communications hardware and software, are known in the art.

The EMR system 200 is specially-configured in accordance with the present invention. Accordingly, as shown in FIG. 2, the EMR system 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the EMR system 200 includes, in accordance with the present invention, a Record Management Engine (RME) 230, shown schematically as stored in the memory 218, which includes a number of additional modules providing functionality in accordance with the present invention, as discussed in greater detail below. These modules may be implemented primarily by specially-configured software including microprocessor—executable instructions stored in the memory 218 of the EMR system 200. Optionally, other software may be stored in the memory 218 and and/or other data may be stored in the data store 224 or memory 218. Further, the RME 230 includes one or more modules shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 2, the improved EMR system 200 includes a data store 224 and a Record Management Engine (RME) 230 in accordance with the present invention. In part, and similarly to convention EMR systems, the improved EMR system 200 stored patient electronic medical record data 224a in the data store 224, e.g., in a database cluster, and the RME 230 includes a Patient Record Module (PRM) 240 that is operable to retrieve electronic medical record data from the patient records 224a, to display the data and prompts for data via a suitable graphical user interface (e.g., on a caregiver computing device 90a, 90b), to receive data that should be added to and stored in a patient's electronic medical record, and to appropriately store the received data as electronic medical record data in the patient records 224a, in a conventional fashion typical of electronic medical records systems. By way of example, the PRM 240 may include a database interface program providing standard database access to one or more database files stored in the data store 224 for this purpose, e.g., using conventional database query language. By way of example, the database files containing the electronic medical record data may be structured data files holding data elements for various data fields associated with relevant medical information, such as, for example, patient height, weight, age, sex, prescription medications, diagnoses, conditions, blood pressure, blood type, heart rate, and any other aspects relevant to medical care.

Additionally, the data store 224 stores additional data, and the RME 230 includes additional modules, in accordance with the present invention. More particularly, the exemplary data store 224 stores shared chart templates 224b in accordance with the present invention. Each of these templates is configured to cause display of a special-purpose graphical user interface (GUI) in accordance with the present invention that acts as a shared chart. The RME 230 includes a Shared Chart Display Module (SCDM) 250 configured to display a GUI acting as a shared chart consistent with corresponding shared chart template 224b, e.g., to display prompts for data via a suitable graphical user interface (e.g., on a caregiver computing device 90a, 90b), to receive data that should be added to and stored in multiple patients' electronic medical records.

Accordingly, the SCDM 250 and a template retrieved from the shared chart template 224b act in concert to cause display of a shared chart GUI that presents data fields for display and/or receiving data elements, somewhat similarly to a conventional EMR GUI window. However, the shared chart GUI includes data fields selected to correspond to a particular clinical workflow involving more than one patient, and thus corresponding to more than one patient's electronic medical record. In other words, the shared chart GUI is process-specific, rather than patient-specific, and is configured to receive and/or display medical record data for both patients, and thus this chart is “shared.” For example, the shared chart template may be configured to correspond to a breastfeeding clinical workflow, which involves more than one patient, namely, a mother and a child. Accordingly, the shared chart template/GUI is configured to include data fields for managing data for both the mother and the child, within a single GUI, and preferably within a single GUI window displayable within a single display screen. Preferably, the data fields are arrangement within the template and/or GUI such that the GUI displays the data fields in an order sequence, or arrangement that matches ore corresponds to the relevant clinical workflow. Accordingly, for example, such a process-specific GUI may prompt for gathering/recording of first data from the first patient/mother, then gathering/recording of second data from the second patient/mother, all within a single GUI window displayed on a single display screen. Thus, data may be gathered and recorded in a sequence that corresponds to a clinical workflow, and may be recorded for multiple patients, without the need to switch between separate electronic medical records or separate data entry windows for separate patients. By way of further example, shared chart templates for GUIs allowing for entry of data for multiple patients via a single GUI/chart may be provided for maternal prenatal lab result information. Such maternal prenatal lab result information is necessary for safe pediatric care for the child, but such prenatal lab result information is not collected and stored as part of the child's chart, as this information does not relate specifically to the child as the child's lab result information, although it is relevant to care for the child. Additionally, maternal delivery care information, such as maternal fever or blood loss, impacts newborn care, such as sepsis evaluation and discharge to home parameters. Accordingly, by way of further example, shared chart templates for GUIs allowing for entry of data for multiple patients via a single GUI/chart may be provided for maternal delivery care information. This information could be entered, and later accessed for reference, via such shared chart templates/GUIs, consistent with the present invention.

The RME 230 and/or the SCDM 250 may be configured to provide a GUI allowing a healthcare provider/operator of the EMR system 200 to select a particular shared chart template and/or corresponding GUI (e.g., the breastfeeding template/GUI) for use. Further, the RME 230 and/or the SCDM 250 may be configured to provide a GUI allowing the healthcare provider/operator of the EMR system 200 to select particular patients (e.g., a particular mother patient and a particular child patient) to associate those patients as patients for which the shared chart template will be used, and the SCDM 250 then stores data as Record Link Data 224c that effectively links those individual patient electronic medical records to each other. Further, the RME 230 and/or the SCDM 250 may be configured to provide a GUI allowing the healthcare provider/operator of the EMR system 200 to select associate a particular shared chart/template/GUI with particular patients (e.g., a breastfeeding shared chart/template/GUI with a particular mother patient and a particular child patient) to associate those particularly shared charts with those patients, and the SCDM 250 then stores data as Shared Chart Associations 224d that effectively link those individual patient electronic medical records to the particular shared chart/template/GUI. Further, this association may be stored automatically by the EMR system 200, e.g., after a particular shared chart/template/GUI is used in association with particular patients. In this manner, for example, the particular mother and child electronic medical record data may be retrieved, displayed and/or accessed via the shared chart. For example, as described above, maternal prenatal lab result information and maternal delivery care information necessary or relevant for care for the child, may be accessed in this manner.

Accordingly, in accordance with the present invention, shared chart functionality is provided that can act as a funnel for data entry of information from the shared chart to individual patient charts, and can act as a “couplet” or group chart for staff to use to browse previously-recorded information. For example, while in the hospital as a couplet/group, staff may access and document relevant information on this shared chart via the shared chart GUI. This avoids problems with current EMR systems, in that they provide for the documenting of care for each patient (mother and baby(ies)) in a vacuum, without regarding to information recorded or needed for the other related patient. After the couplet has been discharged, the shared chart could be split into 2 (or more) individual charts for each person/patient, e.g., for reviewing and reference purposes, if desired. By way of example, a web-based or other patient portal may be provided to allow access so patients can review their care separately from either perspective (mother or child), which can be helpful for when patients move their care, when they are addressing other health concerns or when they are seeking external health opinions.

Accordingly, the EMR system 200 allows a healthcare provider to use a caregiver computing device 90a, 90b to communicate and exchange data with the EMR system to review and/or record clinically-relevant patient information into the shared chart displayed as a shared chart GUI by the RME 200. From the user's perspective, this may appear as data entry, in a generally conventional fashion, within the inventive shared chart GUI. In accordance with the present invention, the data store 224 further stores data sharing rules 224e. The data sharing rules 224e provide a mapping for sharing data among the individual patient electronic medical records 224a and each stored shared chart template 224b. For example, a shared chart template 224b for breastfeeding may include information for causing display of a shared chart GUI including fields for entry of date, time, and quality of feed data. The feed data gathered via the shared chart GUI may pertain to both the mother and the child. For example, such feed data is relevant to the baby in that it speaks to the intake for the newborn, and further is relevant to the mother, e.g., if she is having breast pain, skin breakdown, or having infection symptoms such as mastitis. Accordingly, the data sharing rules 224e provide a mapping such that the feed data is relevant to, and should be stored in, the individual electronic medical record chart of the mother, and also in the individual electronic medical record chart of the child. By way of further example, certain data gathered via the shared chart GUI may pertain only to the mother. Accordingly, the data sharing rules 224e provide a mapping such that the such data is relevant to, and should be stored in, the individual electronic medical record chart of the mother only. Further, other data gathered via the shared chart GUI may pertain only to the child. Accordingly, the data sharing rules 224e provide a mapping such that the other data is relevant to, and should be stored in, the individual electronic medical record chart of the child only. Accordingly, the data sharing rules 224 provide rules for each shared chart template that specifies how data entered into the shared chart template/GUI should be shared with individual patient electronic medical records.

In accordance with the present invention, the RME 230 further includes a Record Synchronization Module (RSM) 260. The RSM 260 is operable to cause data entered via a particular shared chart template/GUI to be shared according to the corresponding data sharing rule 224e for that particular shared chart template/GUI, and to share and store the corresponding shared data in the corresponding patient electronic medical record 224a for each relevant patient, as determined by the record link data 224c associating patients and/or the shared chart associations 224d. Accordingly, from the user's perspective, a healthcare provider may review and/or record clinically-relevant patient information into the shared chart displayed as a shared chart GUI by the RME 200 only once, and the RSM260 automatedly populates relevant data into each relevant patient's electronic medical record to effectively synchronize the individual electronic medical records with the shared chart. In this manner, clinical observations are entered into the EMR system 200 in a streamlined per-process fashion (across multiple patients), rather than in the conventional, segmented per-patient fashion, and the burden on the healthcare provider to enter information into multiple locations, and/or in duplicative fashion, is reduced and/or eliminated.

Further, data relevant to multiple individuals' electronic medical records appears identically in all such records, even though the relevant data is entered only once into the shared chart, as such data is populated into and replicated from a common data source, namely, the shared chart. Accordingly, the electronic medical records of multiple relevant patients are effectively synchronized to each other in that they both contain the same values for the same data elements. In this manner, human error in creating mismatches/data entry errors and/or omissions while entering the same data into multiple patient charts is reduced or eliminated.

Conceptually, the shared chart allows for relevant information to be shared by relevant patient's, so that a separate, per-patient segregation of information in a “sandbox” approach is avoided, but in an appropriate manner. For example, the shared chart may be relevant, and the sandbox approach can be avoided, with respect to safe newborn/breastfeeding education. Currently, many systems are limited such that the documentation of the providing of newborn education is done on the baby chart only, although such education is usually given to the mother, yet on the mother's chart it only shows teaching done about the mother, and not about her newborn. By way of further example, an infant sepsis evaluation needs to have information from the mother chart in order to be completed, and infant immunization protocol is based on the mother's prenatal lab history, such as hepatitis. A linking and combination of mother and child charts indicating the full nature of the education provided, and sharing relevant mother/child information, is relevant to both the mother and to the child. In this manner, the improved system provides increased usability and efficiency for staff, decreasing the double documentation, and further provides for better data gathering for metrics, e.g., for designations such as Keystone 10, proving education tasks are being met. Further, the improved system avoids errors and ensures consistency across charts, which is helpful for auditing and surveys, to ensure that the information placed in one chart be shared into the other chart(s). Further, the ready availability of such relevant, multi-patient information, without the need for search for and review of another patient's chart, tends to avoid pertinent information being overlooked and to improve patient care.

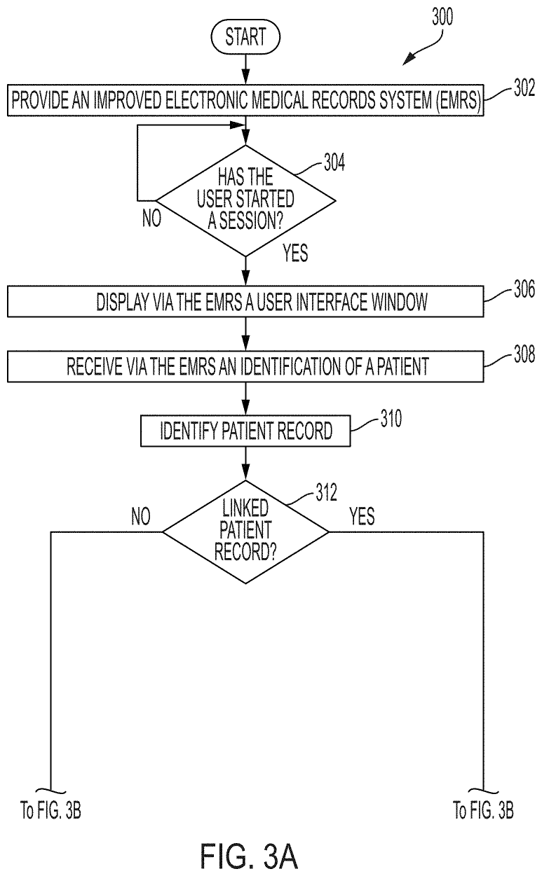

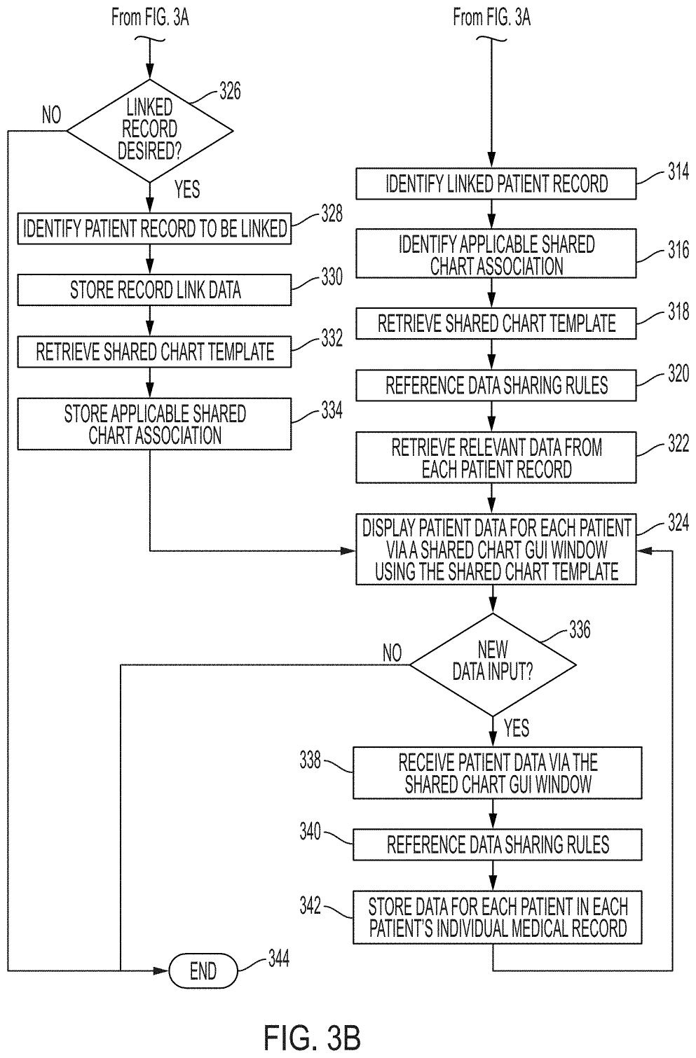

Referring now to FIG. 3, a flow diagram 300 is shown that illustrates an exemplary workflows and methods of operation in accordance with exemplary embodiments of the present invention. As shown in FIG. 3, this exemplary method begins with providing an improved electronic medical records system (EMRS) 200 as shown at 302 of FIG. 3. As discussed above, the EMRS 200 includes a Record Management Engine (RME) 230 and stores particular data in accordance with the present invention, including shared chart templates 224b for providing GUI windows capable of gathering data for multiple related/interconnected patients via a single GUI window, which may be arranged on a per-workflow rather than per-patient basis, patient record linking data 224c identifying individual patient records that are associated/linked, shared chart associations 224d identifying particular charts for multiple patients that are associated/linked, and data sharing rules 224e mapping data relationships for data sharing/cross-population purposes between shared charts/shared chart templates and individual patient records, as described in greater detail above.

In accordance with the exemplary method shown in FIG. 3, the RME 230 next determines whether a user has started an information exchange session with the EMRS 200, as shown at 304. When a session has been started, the RME 230 causes display (e.g., at a Caregiver Computing Device 90a, 90b, 90c) of a suitable graphical user interface (GUI) window allowing interaction with the EMRS, as shown at 306. This GUI window may be any suitable window similar to conventional GUI windows for beginning EMRS data input and/or records search, and allows for input to the EMRS 200 of input identifying a patient.

As shown in FIG. 3, the RME 230 next receives an identification of a patient, e.g., by scanning a patient's data-containing bracelet or receiving user input, e.g., via a mouse or touchscreen at the Caregiver Computing Device 90a, 90b, 90c. The RME 230 then identifies a corresponding patient record from among patient records 224a stored in the data store 224 of the EMRS 200. For example, the RME 230 may identify the patient medical record of Jane Doe, a mother. The RME 230 then determines whether there is a linked patient record, as shown at 312. For example, this may be done be the RME's reference to record link data 224c stored in the data store 224 of the EMRS 200. For example, the RME 230 may identify the patient medical record of John Doe, a child borne by mother Jane Doe in the present hospital stay. The RME 230 then identifies a patient record corresponding to the linked patient, namely, John Doe, as shown at 314, e.g., by reference to the patient record data 224a.

By way of example, the steps described above may be performed and/or managed by the Patient Record Module 240 of the RME 230 of the EMRS 200.

Next, as shown in FIG. 3., the RME 230 identifies applicable shared chart associations for the identified linked patients. For example, there may be one or more shared charts shared by the linked patients, as shown at 316. By way of example, the mother and child referenced above may be associated with a breastfeeding-focused shared chart, as reflected in the shared chart associate data 224d stored in the data store 224 of the EMRS 200.

The RME 230 then retrieves an appropriate shared chart template, as shown at 318, e.g., by retrieving shared chart template data 224b from the data store 224 of the EMRS 200. For example, the RME 230 may retrieve the breastfeeding chart template. The RME 230 references data sharing rules, as shown at 320, e.g., by referencing data share rules data 224e stored in the data store 224 of the EMRS 200. The data sharing rules indicate data rules mapping data fields from the shared chart template to individual patient charts/medical records, as described above. For example, the applicable data sharing rule may provide that certain data retrievable from the mother's chart and certain data retrievable from the baby's chart should be retrieved and displayed in a single shared chart GUI window corresponding to the breastfeeding/shared chart template.

The RME 230 retrieves the relevant data from each patient's (e.g., mother's and baby's) patient record from the patient record data 224, as shown at 322, and then causes display of patient data for multiple patients via a shared chart GUI window using the shared chart template, as shown at 324. For example, this may involve the EMRS 200 transmitting appropriate data via the network 50 to a Caregiver's Computing Device 90a, 90b, 90c.

By way of example, steps 316-324 may be performed and/or managed by the Shared Chart Display Module 250 of the RME 230 of the EMRS 200.

If the EMRS 200 determines that there is no new data input at 336, e.g., in response to a clinician recording with the EMRS 200 new clinical observations, lab results, etc., then this exemplary method ends, as shown at 344. In this manner, a clinician may view patient data that would otherwise be resident in multiple separate patients' charts within a single shared chart and corresponding GUI window, e.g., displayable within a single field of view of a display device of the Caregiver Computing Device, thereby eliminating the need to switch between records of multiple patients.

If, however, the EMRS 200 determines that there is new data input at 336, then the exemplary method continues to receive new data for the patient via the shared chart GUI window (e.g., caused to have been displayed at the Caregiver Computing Device), as shown at 338. By way of example, this may be performed and/or managed by the Shared Chart Display Module 250 of the RME 230 of the EMRS 200.

After new data has been received via the shared chart GUI window, the RME 230 references the data sharing rules, as shown at 340, and then stores data for each patient in each patient's individual medical record, as shown at 342, in accordance with the data sharing rules 224e. For example, this may involve some data entered into the shared chart GUI window being stored in the patient record data 224a for only the mother, or for only the baby, or for both the mother and the baby. By way of example, steps 340 and 342 may be performed and/or managed by the Record Synchronization Module 260 of the RME 230 of the EMRS 200.

In this exemplary method, flow returns to step 324 where patient data, now including the new patient data, is displayed by the shared chart GUI window, and the method continues as shown in FIG. 3. In this manner, data for multiple patients may be entered into a single shared chart GUI window, and the data is automatedly stored and populated into the individual patient records for each patient. This ensures that the relevant records are “in synch”, in that patient data types available in one chart are also available in the other chart, as appropriate, thereby avoiding data omissions from charts. Further, because the data source (the shared chart GUI window) is the same, the same data values are stored and populated automatedly into the individual records, thereby further ensuring that the relevant records are “in synch” in that they contain not only the same types of data, but also the same values for those data types.

Further, the system allows for creating of new shared charts. For example, it if is determined at 312 that there is no linked patient record (e.g., baby patient record) for a first-identified patient record (e.g., mother patient record), then it is determined by the RME 230 if a linked record is desired. If not, this method flow may end, as shown at 344. If however, a linked record is desired, then the RME 230 identifies a patient record to be linked, as shown at 328. For example, this may be done by the patient record module's 240 reference to patient record data 224a in response to user input to the EMRS 200 via a GUI window displayed at a Caregiver Computing Device 90a, 90b, 90c. After another record has been identified with which the first-identified records should be associated, corresponding record link data identifying the association is stored in the record link data 224c, as shown at 330. Further, the RME 230 retrieve's a shared chart template, as shown at 332, e.g., in response to user input via a Caregiver Computing Device indicating that there the user wishes to use a particular, e.g., breastfeeding, template. The RME 230 then stores applicable shared chart association data 224d indicating that a particular shared chart template is associated with the relevant linked patients, as shown at 334. For example, these steps may be done by the shared chart display module 250 in response to user input to the EMRS 200 via a GUI window displayed at a Caregiver Computing Device 90a, 90b, 90c. Flow then continues to receive and display patient data, e.g., as shown at 324 and as described above. In this manner, the improved EMR system provides for reducing the data-entry burden on healthcare providers by avoiding duplicative entry of data for multiple patients, and reduce or eliminates associate data entry errors and omissions, while also ensure that the related (e.g., mother and child) charts are “in sync” and equally up to date, so that data provided in one chart/EMR record is not inadvertently omitted from the other chart/EMR record.

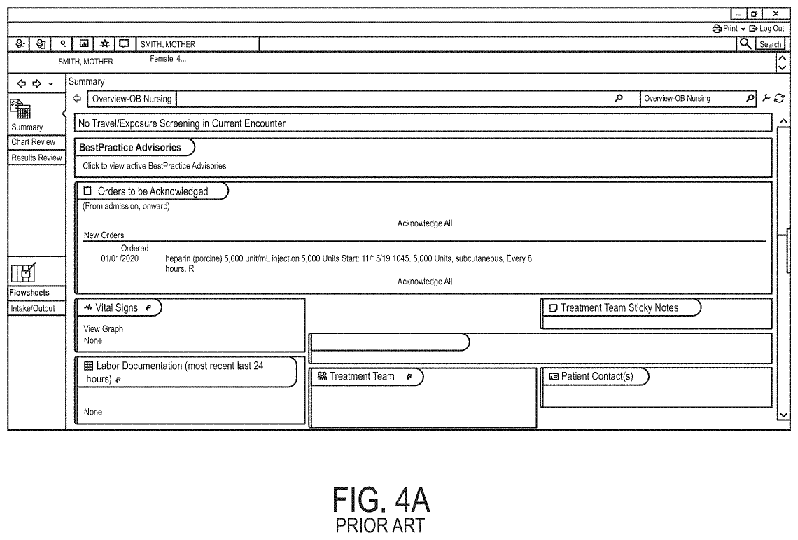

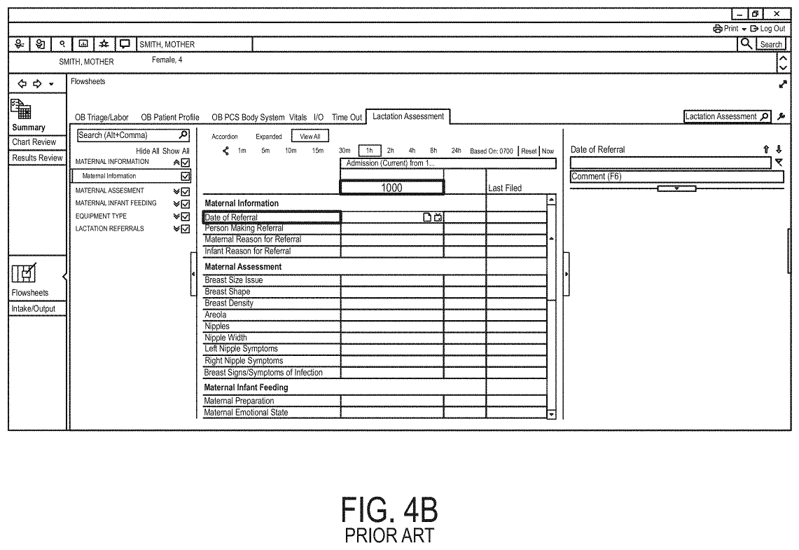

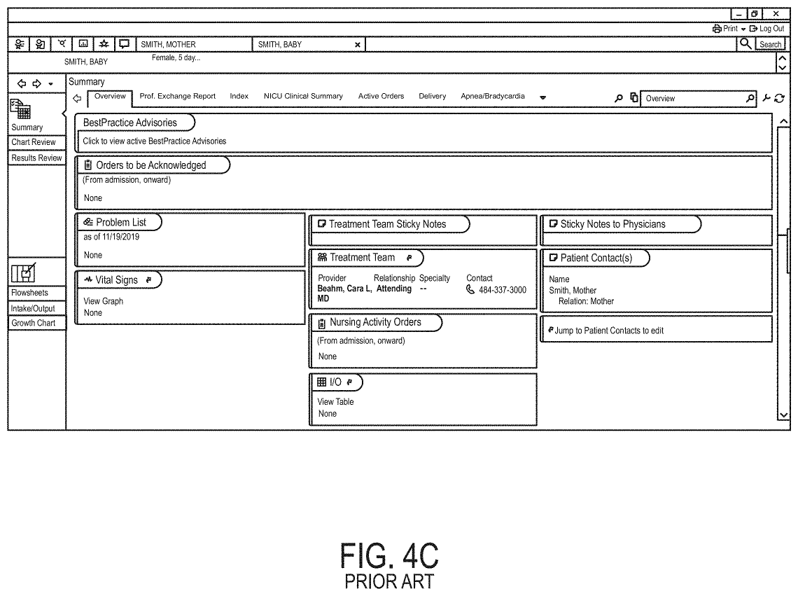

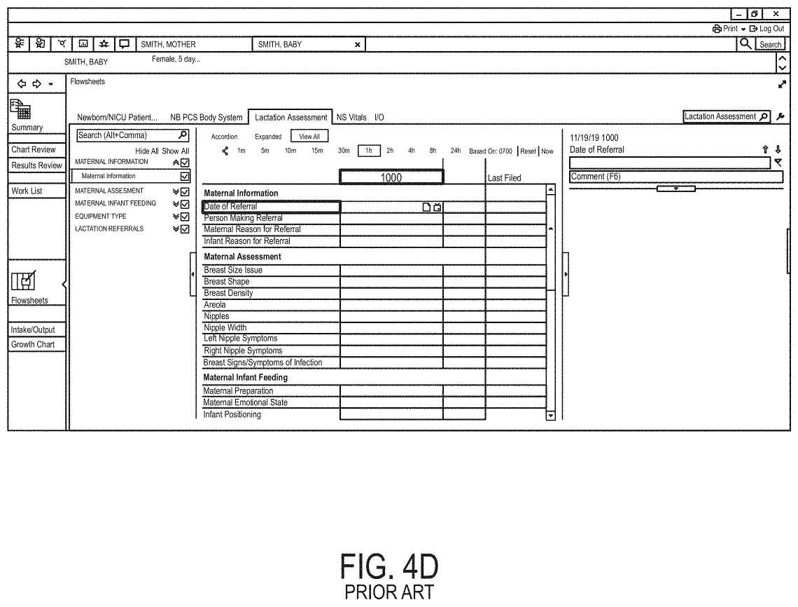

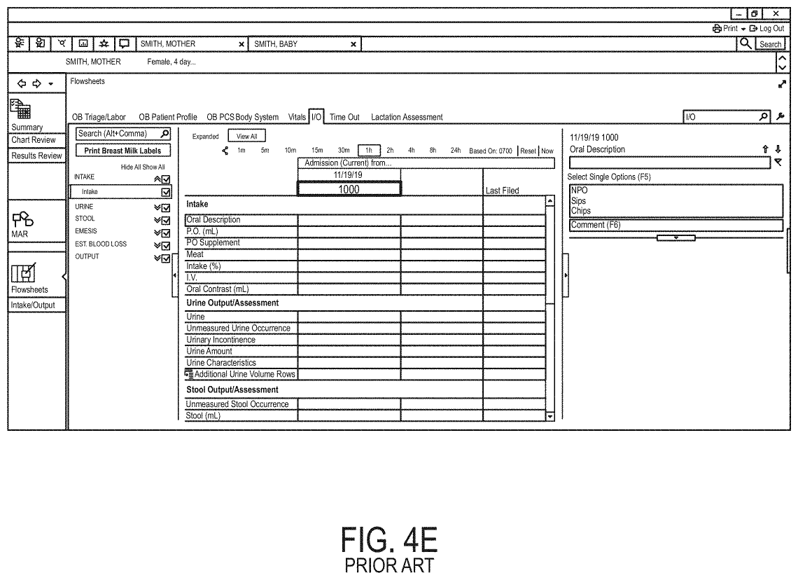

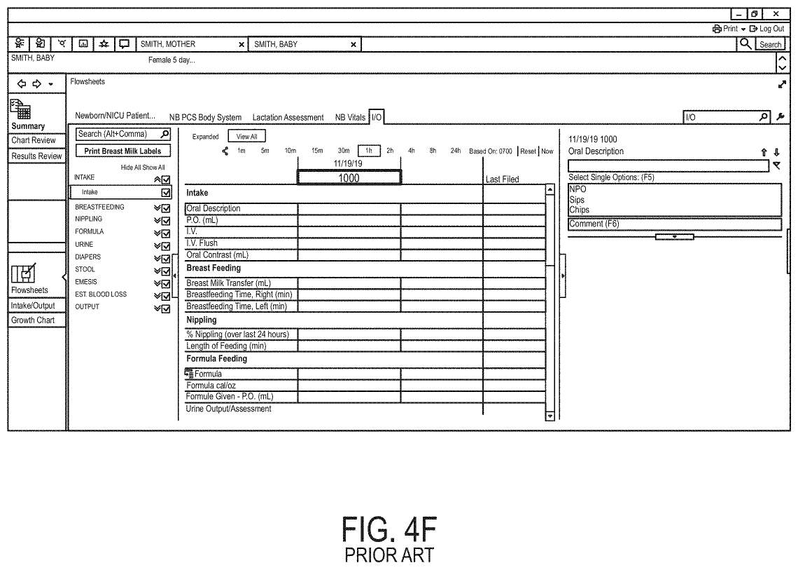

FIGS. 4A-4F are plan views of exemplary graphical user interface windows of a conventional electronic medical records system of the prior art. More particularly, FIGS. 4A, 4B and 4E are graphical user interface windows depicting data fields and elements for receiving and displaying various data values pertinent to a mother's electronic medical record, namely an individual named in this example “Mother Smith”. Somewhat similarly, FIGS. 4C, 4D and 4F are graphical user interface windows depicting data fields and elements for receiving and displaying various data values pertinent to a related patient's electronic medical record, namely the mother's baby, named in this example “Baby Smith”. It will be noted that some of the data fields are identical, and others are related. In the prior art, each of these records (shown in this windows as separate tabs for Mother smith and Baby Smith) are completely separate and distinct records, with no cross-population or data sharing across records performed by the EMR system. Accordingly, the nurse/operator of the system is solely responsible for populating all data fields, which in some cases involves entering the same or similar data twice—once in each record. Further, some of the data elements may be arranged or grouped consistent with clinical workflows, but a single clinical workflow involving both related patients (mother and baby) requires data entry in fields arranged on the separate tabs, which requires switching between tabs, and contributes to the inefficiencies and inaccuracies of the system in gathering and recording data, and thus in inefficient use of the time of the nurse/operator.

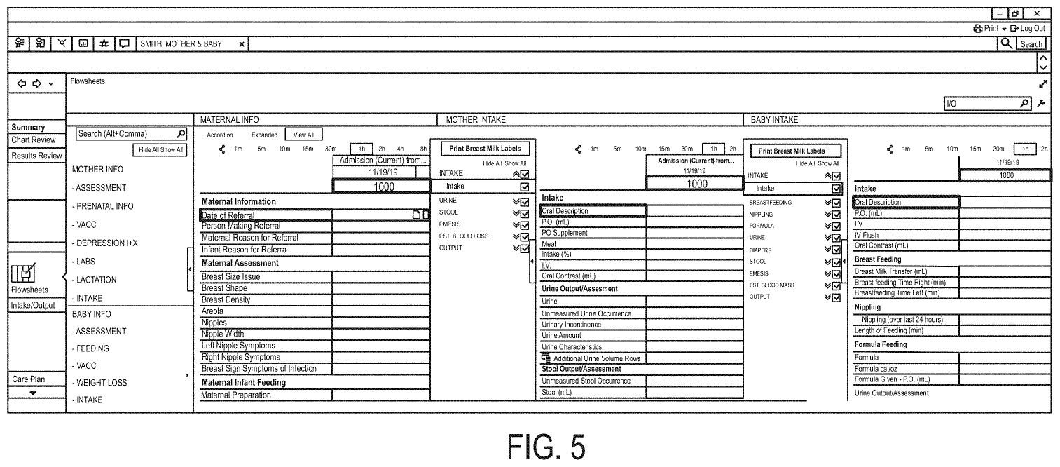

In sharp contrast, FIG. 5 is a plan view of a graphical user interface window of an exemplary graphical user interface window of an improved electronic medical record system in accordance with the present invention. As will be appreciated from FIG. 5, the single exemplary graphical user interface window is displayable on a single physical area of a single display screen/video monitor hardware, and depicts data fields and elements for receiving and displaying various data values pertinent to a multiple patients' medical records, namely the Mother Smith record and the Baby Smith record. Notably, as the result of the record linking tracked by the system, the shared chart templates, shared chart associates, and data sharing rules described herein, data from/for both patients are displayable in this single graphical user interface window. Data from each individual's medical record is combined for viewing purposes, with cross-record data population and data sharing between the mother's and baby's chart as appropriate. Accordingly, data input into the mother's chart and relevant to the baby's chart may be populated into the baby's chart automatically by the system, and vice-versa. Similarly, data retrieved from the mother's chart and relevant to the mother's chart may be displayed in addition to the mother's chart information in a single mother/baby shared chart template, in a single graphical user interface window. Accordingly, the nurse/operator of the system need not enter the same or similar data twice—once in each record. Rather, the nurse/operator can enter data values once into the shared chart template, and the data values may be stored automatically in one or both patients' charts according to the data sharing rules, shared chart associations. Etc. Further, the data elements are arranged or grouped consistent with a single, multiple-patient, clinical workflow involving both related patients (mother and baby). Accordingly, the system allows data entry in fields for both mother and baby/related patients via a single graphical user interface window displayed on a single screen, without the need to switch between tabs/user interface windows for multiple patients. This eliminates inefficiencies and inaccuracies associated with prior art systems in gathering and recording data, and thus in inefficient use of the time of the nurse/operator.

The present invention may be operational with numerous other general-purpose or special-purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, cellular telephones, network PCs, minicomputers, mainframe computers, distributed computing environments that include any of the above-mentioned systems or devices, and the like.

The present invention has been described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer-storage media including, by way of example only, memory storage devices.

The exemplary computing system may include general-purpose computing hardware in the form of a server. Components of the server may include, without limitation, a processing unit, internal system memory, and a suitable system bus for coupling various system components, including a database cluster, with the server. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus.

The server typically includes therein, or has access to, a variety of computer-readable media, for instance, via a database cluster. Computer-readable media can be any available media that may be accessed by the server, and includes volatile and nonvolatile media, as well as removable and non-removable media. By way of example, and not limitation, computer-readable media may include computer-storage media and communication media. Computer-storage media may include, without limitation, volatile and nonvolatile media, as well as removable and non-removable media implemented in any method or technology for storage of information, such as computer readable instructions, data structures, program modules, or other data. In this regard, computer-storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server. Communication media typically embodies computer readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal.

By way of example, and not limitation, communication media includes wired media such as a wired network or direct-wired connection, and wireless media such as acoustic, RF, infrared, and other wireless media. Combinations of any of the above also may be included within the scope of computer-readable media.

The server may operate in a computer network using logical connections to one or more remote computers. Remote computers may be located at a variety of locations or over the Internet. The remote computers may be personal computers, servers, routers, network PCs, peer devices, other common network nodes, or the like, and may include some or all of the elements described above in relation to the server. The computing devices can be personal digital assistants or other like devices.

Exemplary computer networks may include, without limitation, local area networks (LANs) and/or wide area networks (WANs). Such networking environments are commonplace in offices, enterprise-wide computer networks, intranets, and the Internet. When utilized in a WAN networking environment, the server may include a modem/network card or other means for establishing communications over the WAN, such as the Internet. In a networked environment, program modules or portions thereof may be stored in the server, in the database cluster, or on any of the remote computers. For example, and not by way of limitation, various application programs may reside on the memory associated with any one or more of the remote computers. It will be appreciated by those of ordinary skill in the art that the network connections shown are exemplary and other means of establishing a communications link between the computers (e.g., the server and remote computers) may be utilized.

In operation, a user may enter commands and information into the server or convey the commands and information to the server via one or more of the remote computers through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote device to the server. In addition to a monitor, the server and/or remote computers may include other peripheral output devices, such as speakers and a printer.

Many other internal components of the server and the remote computers/computing devices are not shown because such components and their interconnection are well known. Accordingly, additional details concerning the internal construction of the server and the remote computers/computing devices are not further disclosed herein.

Although methods and systems of embodiments of the present invention may be implemented in a WINDOWS or LINUX operating system, operating in conjunction with an Internet-based delivery system, one of ordinary skill in the art will recognize that the described methods and systems can be implemented in any system supporting the search of electronic medical records. As contemplated by the language above, the methods and systems of embodiments of the present invention may also be implemented on a stand-alone desktop, personal computer, cellular phone, smart phone, tablet, PDA, or any other computing device used in a healthcare environment or any of a number of other locations.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A computerized electronic medical record system for cross-patient data population and compact data display comprising:
a memory operatively comprising a non-transitory data processor-readable medium;
a data processor operative connected to the memory; and
record management instructions embodied in data processor-executable code stored in the memory, said record management instructions being executable by the data processor to provide a record management engine configured to cause:
displaying, via a display device of a computing device, of a shared chart graphical user interface window within a physical display area of the display device, the shared chart graphical user interface window being configured to display data values for multiple patients;
retrieving of data values from a first patient electronic medical record and from a second patient medical record; and
displaying, within the shared chart graphical user interface window, of data values retrieved from both the first patient medical record and the second patient medical record within the shared chart graphical user interface window displayed within the physical display area of the display device.

2. The computerized electronic medical record system of claim 1, further comprising:
shared chart template data stored in the memory, each shared chart template defining data fields for data elements from multiple patient data records.

3. The computerized electronic medical record system of claim 2, further comprising:
patient record linking data stored in the memory for each of the plurality of patients, the patient record linking data identifying multiple individual patient records as linked such that data values of one of the first patient electronic medical record and second patient electronic medical record may be relevant to another of the first patient electronic medical record and second patient electronic medical record.

4. The computerized electronic medical record system of claim 3, further comprising:
record synchronization instructions embodied in data processor-executable code stored in the memory, said instructions being executable by the data processor to cause:
receiving of user input of a data value for a data element associated with a first patient electronic medical record of the first patient;
storing of the data value in the memory as part of the first patient electronic medical record; and
storing of the data value in the memory as part of a second patient electronic medical record of the second patient.

5. The computerized electronic medical record system of claim 3, wherein said record synchronization instructions cause storing of the data value in the memory as part of the second patient electronic medical record only if the patient record linking data identifies that the first patient electronic medical record and second patient electronic medical record are linked.

6. The computerized electronic medical record system of claim 3, further comprising:
data sharing rules stored in the memory and mapping data relationships for data sharing across patient records that are linked such that data values of one of the first patient electronic medical record and second patient electronic medical record may be relevant to another of the first patient electronic medical record and second patient electronic medical record.

7. The computerized electronic medical record system of claim 3, wherein said record synchronization instructions cause storing of the data value in the memory as part of the second patient electronic medical record is performed only if the referenced patient record linking data identifies that the first patient electronic medical record and second patient electronic medical record are linked and the data sharing rules identify that a data element for which the data value has been provided is mapped to both the first patient electronic medical record and the second patient electronic medical record.

8. The computer-implemented method of claim 1, wherein the shared chart template is configured to display data elements on the physical display area in an arrangement corresponding to a clinical workflow involving multiple patients.

9. A computer-implemented method for cross-patient data population and compact data display in a computerized electronic medical record system for maintaining electronic medical records for patients, the method comprising:
providing a computerized electronic medical record system for cross-patient data population and compact data display comprising:
a memory operatively comprising a non-transitory data processor-readable medium;
a data processor operatively connected to the memory; and
patient record data stored in the memory for each of a plurality of patients;
shared chart template data stored in the memory, each shared chart template defining data fields for data elements from multiple patient data records;
retrieving of data values associated with a shared chart template from a first patient electronic medical record of the first patient;
retrieving data values associated with the shared chart template from a second patient electronic medical record of the second patient; and
displaying a shared chart template graphical user interface window that displays data retrieved data values from the first patient electronic medical records and the second patient medical record within a physical display area of a display device.

10. The computer-implemented method of claim 9, wherein said computerized electronic medical record system further comprises:
patient record linking data stored in the memory for each of the plurality of patients, the patient record linking data identifying multiple individual patient records as linked such that data values of one of the first patient electronic medical record and second patient electronic medical record may be relevant to another of the first patient electronic medical record and second patient electronic medical record.

11. The computer-implemented method of claim 10, further comprising:
referencing the patient record linking data stored in the memory;
wherein the retrieving data values associated with the shared chart template from the second patient electronic medical record is performed only if the referenced patient record linking data identifies that the first patient electronic medical record and second patient electronic medical record are linked.

12. The computer-implemented method of claim 11, wherein said computerized electronic medical record system further comprises:
data sharing rules stored in the memory and mapping data relationships for data sharing across patient records that are linked such that data values of one of the first patient electronic medical records and second patient electronic medical record may be relevant to another of the first patient electronic medical record and second patient electronic medical record.

13. The computer-implemented method of claim 12, further comprising:
referencing the data sharing rules stored in the memory;
wherein the retrieving data values associated with the shared chart template from the second patient electronic medical record is performed only if the referenced patient record linking data identifies that the first and second patient electronic medical records are linked and the data sharing rules identify that a data element for which the data value has been provided is mapped to both the first patient electronic medical record and the second patient electronic medical record.

14. The computer-implemented method of claim 9, wherein the shared chart template is configured to display data elements on the physical display area in an arrangement corresponding to a clinical workflow involving multiple patients.

15. A computer-implemented method for cross-patient data population and compact data display in a computerized electronic medical record system for maintaining electronic medical records for patients, the method comprising:
providing a computerized electronic medical record system for cross-patient data population and compact data display comprising:
a memory operatively comprising a non-transitory data processor-readable medium;
a data processor operatively connected to the memory; and
patient record data stored in the memory for each of a plurality of patients;
shared chart template data stored in the memory, each shared chart template defining data fields for data elements from multiple patient data records;
displaying a shared chart template graphical user interface window within a physical display area of a display device, the shared chart template graphical user interface window displaying data elements for receiving data associated with both first and second patients;
receiving user input of a data value for a data element associated with a first patient electronic medical record of the first patient;
storing the data value in the memory as part of the first patient electronic medical record; and
storing the data value in the memory as part of a second patient electronic medical record of the second patient.

16. The computer-implemented method of claim 15, wherein said computerized electronic medical record system further comprises:
a patient record linking data stored in the memory for each of the plurality of patients, the patient record linking data identifying multiple individual patient records as linked such that data values of one of the first patient electronic medical record and second patient electronic medical record may be relevant to another of the first patient electronic medical and second patient electronic medical record.

17. The computer-implemented method of claim 16, further comprising:
referencing the patient record linking data stored in the memory;
wherein the storing of the data value in the memory as part of the second patient electronic medical record is performed only if the referenced patient record linking data identifies that the first patient electronic medical record and second patient electronic medical record are linked.

18. The computer-implemented method of claim 17, wherein said computerized electronic medical record system further comprises:
data sharing rules stored in the memory and mapping data relationships for data sharing across patient records that are linked such that data values of one of the first patient electronic medical record and second patient electronic medical record may be relevant to another of the first patient electronic medical record and second patient electronic medical record.

19. The computer-implemented method of claim 18, further comprising:
referencing the data sharing rules stored in the memory;
wherein the storing of the data value in the memory as part of the second patient electronic medical record is performed only if the referenced patient record linking data identifies that the first patient electronic medical record and second patient electronic medical record are linked and the data sharing rules identify that a data element for which the data value has been provided is mapped to both the first patient electronic medical record and the second patient electronic medical record.

20. The computer-implemented method of claim 19, further comprising:
receiving an identification of the first patient as input to the computerized electronic medical record system;
retrieving data from the memory to identify the first patient electronic medical record; and
retrieving data from the memory to reference patient record linking data to identify a second patient electronic medical record linked to the first patient electronic medical record.
